(19) United States
(12) Patent Application Publication
(10) Pub. No.: US 2012/0004625 A1
(43) Pub. Date: Jan. 5, 2012

(54) SPINAL NEEDLE INCLUDING A CHAMBER FOR IDENTIFYING CEREBROSPINAL FLUID

(76) Inventor: Héctor de Jesús Velez-Rivera, Jalisco (MX)

(21) Appl. No.: 12/665,240

(22) PCT Filed: Jun. 4, 2008
(86) PCT No.: PCT/IB08/01431

§ 371 Date: May 17, 2010

(30) Foreign Application Priority Data
Jun. 6, 2007 (MX) MA/A/2007/006751

(51) Int. Cl. A61M 5/00

(52) U.S. Cl. 604/272

(57) ABSTRACT

Disclosed herein is a spinal needle for epidural and spinal anesthesia and analgesia. The needle is formed by three main elements: the first one is a fastening member which allows the user to grab the needle with the fingers; the second element is a chamber that receives an amount of cerebrospinal fluid (CSF); and the third element of the spinal needle is a cannula in fluid communication with the chamber and further attached to the fastening member. The chamber is preferably transparent in order to readily detect the presence of CSF inside the needle. In a preferred embodiment, the chamber has a bottom wall that supports the needle in its entirety. The fastening member and the chamber comprise tactile markings with the same orientation as that of the tip of the cannula, to become aware of the direction in which the anesthetic will be supplied.

### FIELD OF THE INVENTION

The present invention relates to the techniques applied in the designing of devices and equipment for surgical procedures, and more specifically, to a spinal needle including a chamber for cerebrospinal fluid (CSF), wherein the chamber is useful to detect the presence of CSF during anesthesia and analgesia procedures, among other surgical operations.

### BACKGROUND OF THE INVENTION

There exist two basic techniques for introducing medicaments into a patient's spinal area, namely epidural and spinal; these techniques can be employed to create spinal anesthesia or analgesia. In these types of surgical procedures, an anesthetic is injected in the spinal cord and in the nerve roots in order to block pain sensations from one region of the body, such as the abdomen, hips, legs, or pelvis during delivery.

Such medical techniques entail some risks for patients; for such reason, taking into account the main parts of the human body involved in these procedures is important, such as meninges and cerebrospinal fluid that protect the central nervous system. As is well known, meninges comprise three layers: the dura mater, arachnoid and pia mater. Dura mater is the strongest, inflexible and outermost layer thereof; arachnoid is the middle membrane and pia mater is the innermost and delicate layer of the meninges. On the other hand, the cerebrospinal fluid is a clear body fluid that occupies the subarachnoid space, which is the place between the arachnoid and pia mater layers of meninges.

Epidural anesthesia involves the insertion of a hollow spinal needle (for instance, a Tuohy needle), as well as of a flexible catheter into the space between the backbone and the outer membrane of the spinal cord (epidural space) at the middle or lower back. The area where the needle is introduced is blocked with local anesthesia. Then, the needle is inserted and removed after the catheter has gone through the epidural space and said catheter remains in this area. The anesthetic is injected through the catheter so as to block the body area right above or below the point of injection, as needed. The catheter is secured on the top of the back to reuse it, in case more anesthetic is required.

Nevertheless, a problem remains in epidural anesthesia since dura mater and arachnoid layers are so close together that sometimes perforating of the dura mater is not possible without piercing through the arachnoid layer.

Subdural anesthesia is conducted in the same manner, except that the anesthetic is directly injected in the cerebrospinal fluid surrounding the spinal cord with the help of a second spinal needle, such as Whitacre, Quinkle or Sprotte needle; this needle is introduced into the first spinal needle (Tuohy needle). Spinal anesthesia blocks the part of the body right below the site where the anesthetic is supplied, or right above thereof, depending on the anesthetic dose and the technique used to apply the same. In other words, if the tip of the second needle is aimed at the upper part of the body and the anesthetic is dosed, this part of the body will be the one being blocked; the same thing occurs when the anesthetic is applied at the lower part of the body. Sometimes, a spinal catheter may be inserted and left at the place where the injection was applied in order to perform a continuous spinal anesthesia instead of using the second needle.

In fact, any spinal needle must operforate the skin, the subcutaneous fat, the supraspinous ligament, ligaments, the epidural space (in the case of epidural anesthesia|), the dura mater and the arachnoid layer until the needle reaches the subarachnoid space, wherein the spinal cord and nerve roots are located and surrounded by the CSF (in the case of the spinal anesthesia).

On the other hand, there also exists the combined anesthesia, consisting of the application of both epidural and spinal anesthesia. The first one is employed in surgery and the other one is used for additional doses during analgesia while in surgery and post-surgery.

Methods and devices have been developed in the prior art to locate the epidural or subarachnoid space, such as the method disclosed in the U.S. Pat. No. 6,925,323, which deals with a system to increase visibility of the epidural space, wherein such document discloses an epidural surgery method that improves visibility of the epidural space in a patient, with the purpose of efficiently performing therapeutic surgery at such space. The method includes the steps of distending a portion of the epidural space by filling the portion of the epidural space with a fluid supplied from a catheter; placing an optical gap in the distended portion of the epidural space by inserting the optical gap through the same catheter that supplies the fluid for allowing the distension, thereby allowing a visual image of the epidural space.

U.S. Pat. No. 6,773,417 discloses a epidural space locating device comprising a body section having a first end and a second end; a longitudinal passageway extending therethrough, wherein the first end is couplaeble to a luer assembly; and, a collapsible rear chamber having one end coupled to the second end of the body section and the other end is exposed so as to allow pressure to be exerted thereon by one or more fingers of the hand, such that when positive pressure exists within the chamber, it maintains its shape, whereas when negative or zero pressure exists within the chamber, the latter collapses and thus indicates the location of the epidural space by a needle that is coupled to the luer assembly; and the loss of pressure within the chamber is sensed by the fingers of the hand as the shape of the chamber collapses.

In epidural anesthesia or in epidural, ensuring that the injection correctly perforates through the desired area is very important; otherwise, the anesthetic can affect the nervous, cardiovascular and respiratory systems. Both spinal and epidural anesthesia may significantly affect breathing, heartbeats and other vital functions. Furthermore, there is a potential risk of toxicity caused by large doses of medicament, unnecessary to achieve a proper blockade. As mentioned above, the flow direction in which the anesthetic is dosed in the epidural or subarachnoid space is very important to achieve the objective of blocking the desired part of the body.

In connection with the above, U.S. Pat. No. 6,558,353 discloses a needle comprised of a needle hub disposed at the proximate end of a hollow needle. The needle hub has port indicators that provide visual and tactile verification to a user regarding the orientation of the tip of the needle. and more specifically, the needle hub includes projections for tactile verification and a magnifying window for visual verification. However, both the projections and the window are very small and the visual detection of CSF through the needle is not always achieved. In fact, in some instances, small air bubbles gather below the window, thereby blurring the view when a user looks therethrough.

Additionally, Whitacre and Tuohy needles, which are known from the prior art, include a small slot that aids the surgeon in detecting orientation of the tip of the needle; however, in this type of simple needles as well as in that of U.S. Pat. No. 6,925,323, the presence of CSF through the needle has not been effectively detected in a visual manner. The magnifying window of U.S. Pat. No. 6,925,323 is very small when compared with the needle hub. A common medical practice to detect CSF has been to allow the dripping of CSF at the proximate end of the spinal needle; however, if an excessive loss of CSF occurs, the patient suffers from headaches. For this reason, the presence of CSF must be clearly detected in order to dose the medication or the anesthetic at the proper place and in the correct direction and location of the meninges; otherwise, if no CSF can be obtained by means of the needle, the surgical procedure has to be called off.

Although some devices and methods exist to locate the epidural or subarachnoid space, there still remains the drawback of visually detecting CSF through the spinal needles.

### SUMMARY OF THE INVENTION

In order to overcome prior art issues regarding tip orientation of the spinal needs and viewing of CSF, a solution has been devised, which is particularly related to a spinal needle comprising: a fastening member with a proximate end and a distal end and having a first tactile marking formed on its outer surface; one chamber protruding around the fastening member and receiving the CSF therein, said chamber being transparent to visually detect the presence of CSF therein; and, a cannula connected to the distal end of the fastening member and in fluid communication with the chamber. To know the orientation of the tip of the cannula, the latter is oriented in the same direction with regard to the first tactile marking.

The combination of these elements forms a spinal needle with chamber, which is employed to detect the presence of CSF through the needle during anesthesia and analgesia procedures, among other surgical operations.

In a preferred embodiment of the invention, the chamber has a bottom wall in a shape such that it supports the needle at a stable position on a surface. In another preferred embodiment of the invention, the chamber comprises a second tactile marking oriented in the same direction with regard to the tip of the cannula.

In view of the above, it can be said that one object of the present invention is to provide a spinal needle with a chamber in order to clearly detect the presence of CSF through a spinal needle.

### BRIEF DESCRIPTION OF THE DRAWINGS

The novel featuring aspects of the present invention will be set forth in particular in the appended claims. Nevertheless the invention itself, both in its structure and performance, and along with other objects and advantages thereof, shall be better understood in the following detailed description of a preferred embodiment, when read in connection with the accompanying drawings, wherein:

FIG. 1 is a perspective view of a spinal needle with a chamber to identify CSF, builded according to one preferred embodiment of the present invention.

FIG. 2 is a right side view of the needle displayed in FIG. 1.

FIG. 3 is an upper plant view of the needle displayed in FIG. 2.

FIG. 4 is a view of the proximate end of the needle displayed in FIG. 1.

FIG. 5 is a cross section view taken alongside line A-A′ of FIG. 1.

FIG. 6 is a side view of a cross section according to FIG. 5.

FIG. 7 is an upper plant view showing the coupling between the needle of FIG. 1 and a Tuohy-type needle.

### DETAILED DESCRIPTION OF THE PREFERRED EMBODIMENTS

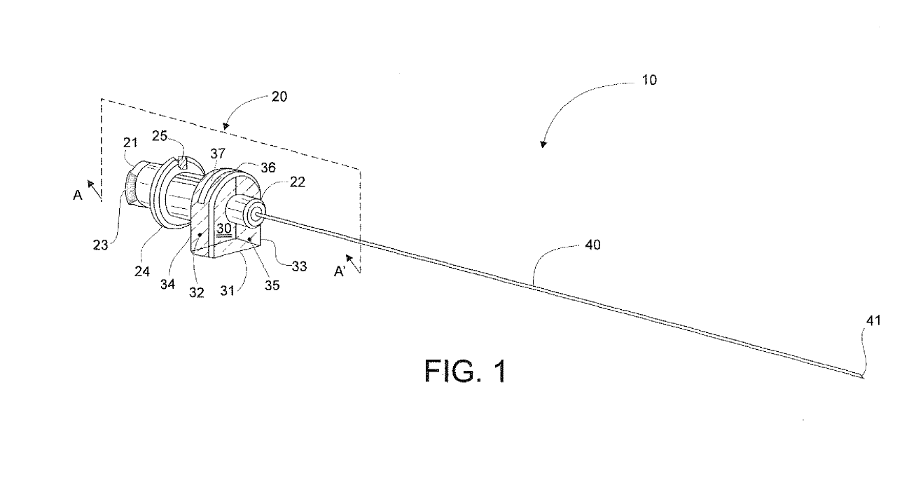

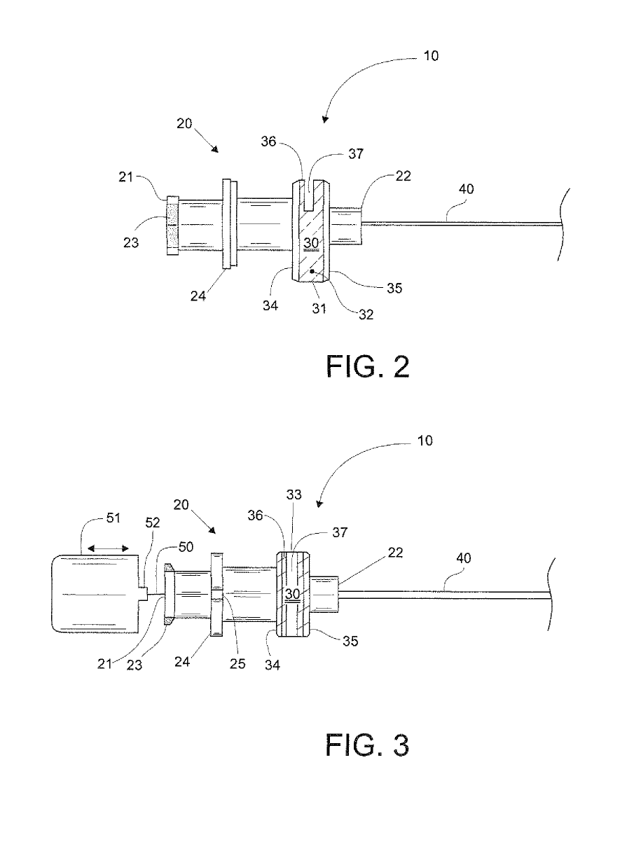

Referring to FIGS. 1 to 4, there is shown therein a spinal needle with a chamber for the CSF, the needle being identified with number 10 and employed during epidural and spinal surgical procedures. The spinal needle with chamber 10 is designed according to the principles of a first preferred embodiment of the present invention, which is to be considered as illustrative rather than limiting thereof. In FIGS. 2 and 3, the distal end of the spinal needle 10 is not shown for clarity purposes. According to medical practice, the distal direction is that closer to the patient, whereas proximate direction is that oriented towards the physician.

From FIGS. 1 to 4, it can be seen that the spinal needle 10 comprises three main elements, the first one is a fastening member 20 that allows a user to hold the needle 10 with the fingers; the second element is a chamber 30 that receives a small amount of CSF that, according to the preferred embodiment, has a capacity of about 0.001 to approximately 10 milliliters; and the third main element of the spinal needle 10 is a cannula 40 in fluid communication with the chamber 30 and attached to the fastening member 20, the cannula being introduced into the patient's back to begin with the anesthesia procedure.

The fastening member 20 has a proximate end 21 and a distal end 22, of which the proximate end 21 includes a port to connect a medical fluid management device thereto, such as a syringe or a catheter containing a medicament or an anesthetic therein to be dosed. In the preferred embodiment being described, said port is particularly provided in the shape of a “luer” type connector 23; however, other types of connectors may be employed for the same objective. Luer connectors are widely used in the medical field and no need to describe them further herein is required.

On the outer surface and at the middle portion of the fastening member 20, a protruding ring 24 is integrally formed, this ring 24 including a first tactile marking in the shape of a first slot 25 shaped in the upper part of the protruding ring 24 and extending longitudinally thereon; the first slot 25 can be touched by the index finger of the user in order to become aware of the orientation of the tip 41 of the cannula 40 due to the fact that the tip 41 and the first slot 25 are in the same orientation. The tip 41 of the cannula 40 has a pencil-style tip, well known in the prior art. Other types of tips may be used for such cannula 40, such as quinkle- and sprotte-type tips.

In order to avoid the introduction of a foreign material inside the needle 10 prior to its use, a filament 50 is employed inside the cannula 40, the filament 50 being attached to a hollow plug 51, which includes a tooth 52 that is coupled inside the first slot 25. In this regard, when coupling the tooth 52 inside the slot 25, the plug 50 encircles a portion of the proximate end of the fastening member 20 and the luer type connector 23 to protect the same.

On the other hand, the chamber 30 protrudes around the fastening member 20 and is located close to the distal end 22, although it may also be close to the proximate end 21 of the fastening member 20. For manufacturing purposes, the chamber 30 is integrally formed with the fastening member 20 and in fluid communication with the cannula 40, such that when the subarachnoid or epidural space has been located, the CSF flows from the tip 41 of the cannula 40 towards the chamber 30, wherein a small amount of CSF is collected, typically from 0.001 to about 10 milliliters, this amount neither adversely affects the dosing rate of the anesthetic nor causes inconvenience on the patient, as CSF is not lost. The chamber 30 is substantially transparent so as to visually detect the presence of CSF when the same is inside the chamber.

In addition, the chamber 30 has a flat bottom wall 31; left and right walls 32 and 33 respectively; a proximate wall 34; a distal wall 35; and an upper wall 36; said chamber 30 has a second tactile marking with the shape of a second slot 37 provided on the upper wall 36, said second slot extending over the upper wall 36 from the right wall 32 towards the left wall 33, i.e. extending in transversal direction, and is notoriously longer than the first slot 25, and therefore can be easily seen by the user and touched with the index finger. The upper wall 36 may have a flat, concave or convex shape.

Over the surface of the right and left walls 32 and 33 respectively, a user may put his fingers to grab the needle and introduce the same, for instance, into a Tuohy-type needle. Usually the middle finger and the thumb are those employed to grab the spinal needle 10, whereas the index finger is used to locate the first slot 25 or the second slot 37.

The bottom wall 31 is particularly important because it serves as a base or platform to totally support the spinal needle 10 on any flat surface in a stable position. In the prior art, the spinal needles roll due to the cylindrical shape of the hub or grabbing portion thereof. This issue is overcome in the present invention thanks to the flat bottom wall 31 of the chamber 30. In addition, when the spinal needle 10 is supported by said flat bottom wall 31 the user may rapidly grab the spinal needle and become aware of the orientation of the tip 41 of the cannula 40 in advance, since the flat wall 31 is opposite to the second slot 37. Other geometric shapes for the bottom wall 31 can be provided, such as a convex shape, a gabled shape, a flat shape with a plurality of ridges, etc. as long as these allow the spinal needle to remain at a stable position.

Regarding the cannula 40, it is attached to the distal end 22 of the fastening member 20 and in fluid communication with the chamber 30, said cannula 40 being very thin. This feature is due to its design to perforate the dura mater, thereby reducing the possibility that the patient suffers from headaches after the surgery; additionally, the cannula 40 provides an accurate access and greater control for the patient's safety and comfort. The tip 41 of the cannula is preferably of the pencil-tip type and has the same orientation than that of the first slot 25 of the fastening member 20 and of the second slot 37 of the chamber 30.

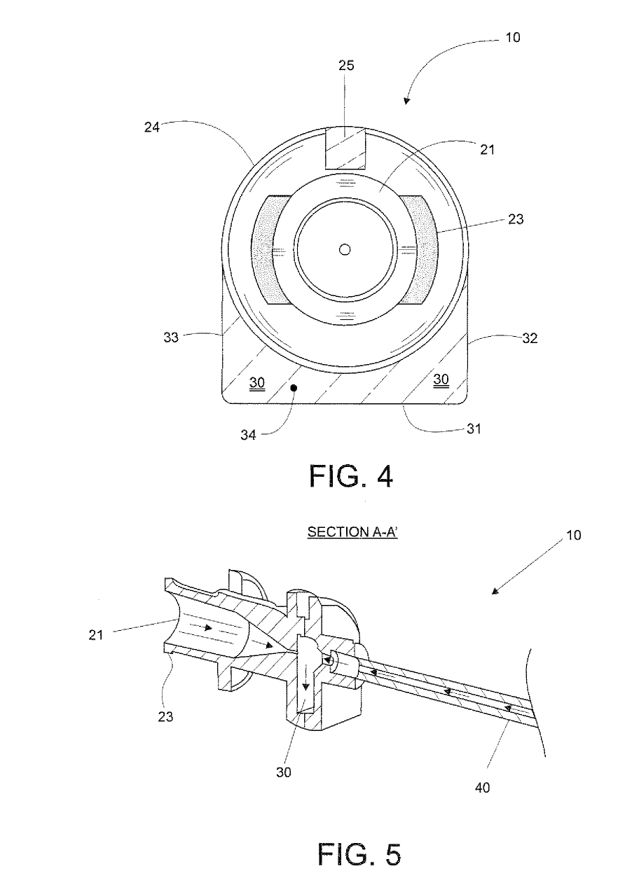

FIG. 4 shows a view of the proximate end of the spinal needle 10, wherein the luer connector 23 can be readily viewed; behind said connector 23 the protruding ring 24 and the first slot 25 just at the upper part of the protruding ring 24 can be seen; and the chamber 30 with the flat bottom wall 31 that supports the spinal needle. In this figure, the right 31 and left 32 walls, which are used to hold the spinal needle 10, are also identified.

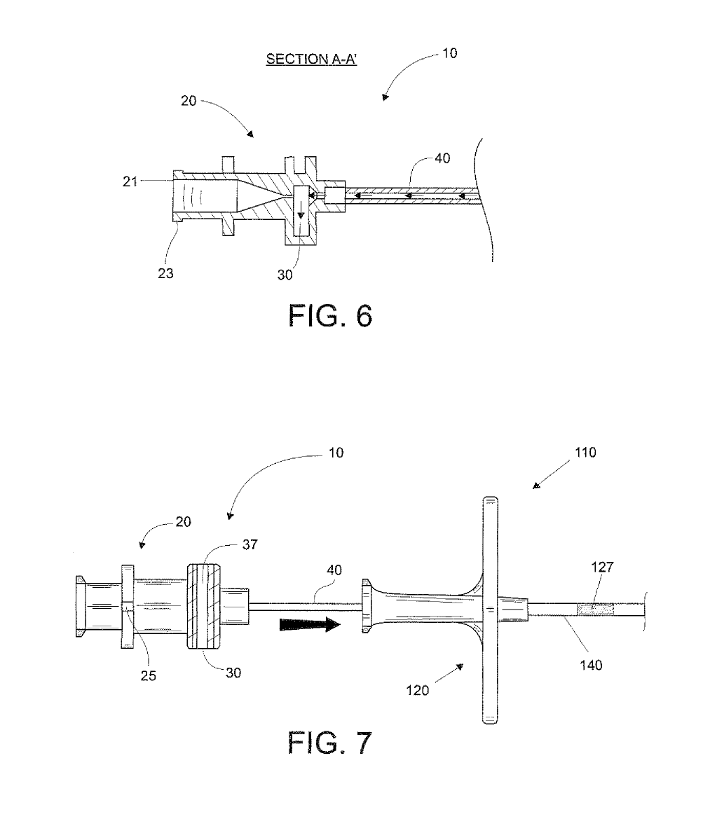

Now, reference is made to FIGS. 5 and 6 to explain how CSF flows through the spinal needle 10. When the user has located the subarachnoid space, CSF flows through the interior of the cannula 40 towards the chamber 30; wherein a small amount of CSF is collected; then, the CSF can be easily viewed from any of the chamber 30 walls. Once the user is sure that he has found the subarachnoid space upon presence of CSF in the chamber 30, the user may couple the medical fluid management device to the luer connector 23 of the proximate end 21 from the fastening member 20, and afterwards, the surgeon doses the anesthetic or the medicament through the spinal needle 10. The chamber 30 does not affect dosage of the anesthetic or of the medicament.

Finally, FIG. 7 shows how the spinal needle 10 of the preferred embodiment of this invention is coupled to a Tuohy needle 110, which has a fastening member 120 and a cannula 140. The Tuohy needle 110 is used to locate the epidural space and then the spinal needle 10 may be introduced to effectively detect the presence of CSF that can be viewed through the chamber 30 walls. During the coupling between both needles 10 and 110, the cannula 40 of the spinal needle 10 is introduced longitudinally through the cannula 140 of the Tuohy needle until the distal part 22 of the spinal needle 10 is inside the fastening member 120 of the Tuohy needle 110. The cannula 140 has some markings 127 that indicate how deep the cannula 140 has been introduced into the patient's tissues. The slots 25 and 37 allow the user to become aware of the orientation of the tip of the cannula 40, regardless of the tip 41 being inside the Tuohy needle.

Prior-art Tuohy, Whitacre, Quinkle or Sprotte needles may be configured according to the principles of the present invention; i.e., the spinal needle chamber of the present invention may be furnished in such well known type of needles.

Although a preferred embodiment of the present invention has been disclosed and illustrated, it should be emphasized that numerous modifications can be made thereto, such as the geometric shape of the fastening member, the shape of the chamber, and so on. Therefore, the present invention is not be regarded as limited except for the requirements of the prior art and by the appended claims.

1. A spinal needle with a chamber to identify cerebrospinal fluid (CSF), characterized because it comprises:
a) a fastening member with a proximate end and a distal end and having a first tactile marking formed on its outer surface;
b) a chamber protruding around said fastening member and receiving CSF; said chamber being transparent to visually detect the presence of CSF in said chamber; and,
c) a cannula attached to the distal end of said fastening member and in fluid communication with said chamber, the tip of the cannula being oriented in the same direction regarding the first tactile marking.

2. A spinal needle with a chamber to identify cerebrospinal fluid, according to claim 1, further characterized because said chamber has a bottom wall supporting the spinal needle in a stable position on a surface.

3. A spinal needle with a chamber to identify cerebrospinal fluid, according to claim 2, further characterized because the bottom wall has a flat or convex surface.

4. A spinal needle with a chamber to identify cerebrospinal fluid, according to claim 1, further characterized because said chamber has right and left walls, wherein a user may place his/her fingers to grab the needle.

5. A spinal needle with a chamber to identify cerebrospinal fluid, according to claim 1, further characterized because said chamber is located at the distal end of said fastening member.

6. A spinal needle with a chamber to identify cerebrospinal fluid, according to claim 1, further characterized because said chamber is located at the proximate end of said fastening member.

7. A spinal needle with a chamber to identify cerebrospinal fluid, according to claim 1, further characterized because said chamber has a capacity of 0.001 milliliters to 10 milliliters.

8. A spinal needle with a chamber to identify cerebrospinal fluid, according to claim 1, further characterized because said fastening member has a ring radially protruding therefrom and including the first tactile marking.

9. A spinal needle with a chamber to identify cerebrospinal fluid, according to claim 8, further characterized because the first tactile marking is a slot formed at the uppermost part of the ring and extending on the ring longitudinally.

10. A spinal needle with a chamber to identify cerebrospinal fluid, according to claim 1, further characterized because said chamber includes a second tactile marking oriented in the same direction with regard to the tip of said cannula.

11. A spinal needle with a chamber to identify cerebrospinal fluid, according to claim 10, further characterized because said chamber has an upper wall where the second tactile marking is formed.

12. A spinal needle with a chamber to identify cerebrospinal fluid, according to claim 10, further characterized because said second tactile marking is a second slot extending over the upper wall in transversal direction.

13. A spinal needle with a chamber to identify cerebrospinal fluid, according to claim 12, further characterized because the upper wall has a flat, concave or convex shape.

14. A spinal needle with a chamber to identify cerebrospinal fluid, according to claim 1, further characterized because said fastening member has a port at its proximate end to connect a medical fluid device.

15. A spinal needle with a chamber to identify cerebrospinal fluid, according to claim 14, further characterized because said port is a luer-type connector.

16. A spinal needle with a chamber to identify cerebrospinal fluid, according to claim 1, further characterized because said cannula has a tip selected from the group consisting of a pencil-type, a quinkle-type or a sprotte-type tip.
